(19) United States
(12) Patent Application Publication
(10) Pub. No.: US 2026/0256366 A1
(43) Pub. Date: Sep. 3, 2026

(54) BLOOD PRESSURE MONITORING SYSTEM

(71) Applicant: KONINKLIJKE PHILIPS N.V., EINDHOVEN (NL)

(72) Inventors: RICHARD EARL GREGG, WESTFORD, MA (US); BRIAN DAVID GROSS, NORTH ANDOVER, MA (US); WEI ZONG, WOBURN, MA (US)

(21) Appl. No.: 18/714,289

(22) PCT Filed: Nov. 30, 2022
(86) PCT No.: PCT/EP2022/083767

§ 371 Date: May 29, 2024

Related U.S. Application Data
(60) Provisional application No. 63/284,202, filed on Nov. 30, 2021.

(51) Int. Cl. A61B 5/0215; A61B 5/00; A61B 5/021

(52) U.S. Cl. CPC A61B 5/0215; A61B 5/02108; A61B 5/7264; A61B 5/7278; A61B 5/7405; A61B 5/742; A61B 5/7455; A61B 5/746

(57) ABSTRACT

One or more systems, computer implemented methods, and apparatus to dynamically monitor blood pressure (BP) performance and, in response to detection of a poor dynamic response, automatically initiate a flush sequence of one or more flushes and/or automatically generate one or more alerts, alarms, or warnings (e.g., visual, audio, haptic, etc.) to one or more medical professionals indicating the types of issue(s) directly or indirectly causing to the detected poor dynamic BP response.

### TECHNICAL FIELD

One or more systems, computer-implemented methods, and apparatus to dynamically monitor blood pressure (BP) performance by detecting an invasive BP dynamic performance, facilitating the troubleshooting of bad dynamic measurements, and displaying an estimated blood pressure waveform with an indication of a range of the true dynamic waveform values given the detected invasive BP dynamic performance.

### BACKGROUND

Invasive arterial (intra-arterial) blood pressure (IABP) monitoring is a commonly used technique in an intensive care unit (ICU) that is also used in the operating room, when hemodynamic instability is a risk or when beat-to-beat measurements and visualization of the pressure waveform are helpful.

In contemporary clinical practice, IABP monitoring is usually conducted via a tubing system which involves the insertion of a catheter, e.g., into a suitable artery (e.g., radial artery) which is connected to an external pressure sensor. The tubing system for IABP monitoring may comprise an intra-arterial cannula, a catheter, a pressure transducer, one or more stopcocks or valves, flush tubing, and a pressure signal processing unit that includes a display. The arterial blood pressure (ABP) is transmitted from the artery through a column of non-compressible, bubble free fluid (0.9% saline) in the catheter to the pressure transducer. The flush tubing includes a bag of the saline which is pressurized to 300 mmHg and attached to the fluid-filled tubing via a flush system. The flush system allows a high-pressure flush of fluid through the system in order to keep the catheter clear and to check the dynamical characteristics (e.g., damping and natural frequency) of the system.

With necessary initial settings of the tubing system, the IABP monitoring may be performed continuously for hours or even days. Accordingly, during such continuous IABP monitoring, a clot may form at the catheter tip. This will result in damping of the pressure transmitted to the sensor, which in turn, results in a dampened pressure signal. There are other factors which cause ABP damping: the presence of air bubbles in the catheter, kinking of the pressure tubing due to the use of excessively long pressure tubing, movement of the catheter to a location where fluid movement is impeded, or blood flow into the catheter. The damped ABP signal will cause inaccurate blood pressure (BP) measurements (especially the systolic and diastolic BP), leading to false ABP alarms and/or causing misinterpretation of a hemodynamic situation.

A medical clinician or professional conducting the IABP monitoring may become aware of ABP damping by visually observing the ABP waveform on the display. When ABP damping is suspected, quick action must be taken to conduct a fast flush of the catheter, which allows the pressurized saline to flow through the catheter tubing for a short period of time (e.g., between 2 to 3 seconds), which serves to clear the catheter. After conducting the fast flush, IABP monitoring will continue should the damped ABP signal return to normal dynamic status. Otherwise, one or more supplemental fast flushes may be required. In situations where multiple fast-flushes do not resolve the dampened ABP signal, the catheter may be replaced by a new set of pressure tubing.

An ABP damping event is an abnormal measuring condition in IABP monitoring. Because the ABP damping event is manually observed/identified, it can be overlooked or misidentified with delays due to human error and/or fatigue. Moreover, because a flushing operation is conducted manually, related clinical workflow is negatively impacted and clinical workload is increased. Thus, an ABP damping event should be quickly identified and resolved to avoid an incorrect interpretation of the patient hemodynamic condition.

### SUMMARY

One or more embodiments relate to one or more systems, computer-implemented methods, and apparatus to dynamically monitor BP performance of a system by measuring IABP dynamic performance, facilitating the troubleshooting of bad dynamic measurements, and visually displaying an estimated BP waveform with an indication of a range of the true dynamic waveform values given the measured IABP dynamic performance.

In accordance with one or more embodiments, one or more systems, computer implemented methods, and apparatus includes dynamically monitoring of an IABP measurement and, in response to detection of a poor dynamic response, automatically initiate a first flush sequence of one or more flushes and/or automatically generate one or more alerts, alarms, or warnings (e.g., visual, audio, haptic, etc.) to one or more medical professionals indicating the types of issue(s) directly or indirectly causing to the detected poor dynamic BP response.

In accordance with one or more embodiments, a measured dynamic response of a peripheral arterial catheter (“A-line”) BP measurement system is used to indicate a possible range of a true signal.

Troubleshooting can be extended to correlation of non-invasive arterial BP (NIABP) with IABP. Low BP alarms, alerts, or messages can be qualified with the automated NIABP thus, saving the medical professional the time to respond to the BP alarm and initiate a NIABP measurement.

To troubleshoot dynamic measurement problems, the diagnosis starts with conducting a square wave test to measure the dynamic accuracy of the IABP measurement. The BP signal responds to the square wave test, a fast flush, as a step response. The square wave test indicates whether the measurement system is damped correctly or overdamped or underdamped. Over damping is the most common problem resulting in a measured dynamic pressure that is not as high or low as the true dynamic pressure.

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein. It should also be appreciated that terminology explicitly employed herein that also may appear in any disclosure incorporated by reference should be accorded a meaning most consistent with the particular concepts disclosed herein.

In accordance with one or more embodiments, a system comprises one or more of the following: an IABP monitoring apparatus; and a BP monitor operatively connected to the IABP apparatus, the BP monitor including one or more processors and a non-transitory memory operatively coupled to the one or more processors comprising a set of instructions executable by the one or more processors to cause the one or more processors to: initiate a first flush sequence of one or more flushes of a catheter of the IABP apparatus; dynamically detect a BP waveform of an arterial BP signal generated by the IABP monitoring apparatus; and dynamically detect, responsive to the first flush sequence, presence of damping in the detected BP waveform by applying a machine learning algorithm to conduct an analysis of the detected BP waveform which appears as a square wave stimulus.

In accordance with one or more embodiments, a computer-implemented method of dynamically monitoring a BP measurement comprises one or more of the following: initiating a first flush sequence of one or more flushes of a catheter of an IABP monitoring apparatus; dynamically detecting a BP waveform of an arterial BP signal generated by the IABP monitoring apparatus; and dynamically detecting, responsive to the first flush sequence, presence of damping in the detected BP waveform by applying a machine learning algorithm to conduct an analysis of the detected BP waveform which appears as a square wave stimulus.

In accordance with one or more embodiments, an apparatus comprises one or more of the following: a BP monitor including one or more processors and a non-transitory memory operatively coupled to the one or more processors comprising a set of instructions executable by the one or more processors to cause the one or more processors to: initiate a first flush sequence of one or more flushes of a catheter of an IABP monitoring apparatus; dynamically detect a BP waveform of an arterial BP signal generated by the IABP monitoring apparatus; and dynamically detect, responsive to the first flush sequence, presence of damping in the detected BP waveform by applying a machine learning algorithm to conduct an analysis of the detected BP waveform which appears as a square wave stimulus.

These and other aspects of the various embodiments will be apparent from and elucidated with reference to the embodiment(s) described hereinafter.

### DRAWINGS

The various advantages of the embodiments will become apparent to one skilled in the art by reading the following specification and appended claims, and by referencing the following drawings, in which:

FIG. 1 illustrates a block diagram of an example BP monitoring system, in accordance with one or more embodiments set forth, shown, and described herein.

FIG. 2 illustrates a block diagram of an IABP monitoring apparatus of the blood pressure diagnostic system of FIG. 1, in accordance with one or more embodiments set forth, shown, and described herein.

FIG. 3 illustrates a block diagram of an example BP monitor of the BP monitoring system of FIG. 1, in accordance with one or more embodiments set forth, shown, and described herein.

FIG. 4 illustrates a block diagram of a dynamic response measurement of the BP monitoring system of FIG. 1, in accordance with one or more embodiments set forth, shown, and described herein.

FIG. 5 illustrates a block diagram of an estimation of a true waveform of the BP monitoring system of FIG. 1, in accordance with one or more embodiments set forth, shown, and described herein.

FIGS. 6 and 7 respectively illustrates an example display of example of an IABP damping detection and flush effectiveness assessment, in accordance with one or more embodiments set forth, shown, and described herein.

FIGS. 8 and 9 respectively illustrate a flowchart of an example computer-implemented method, in accordance with one or more embodiments set forth, shown, and described herein.

### DETAILED DESCRIPTION

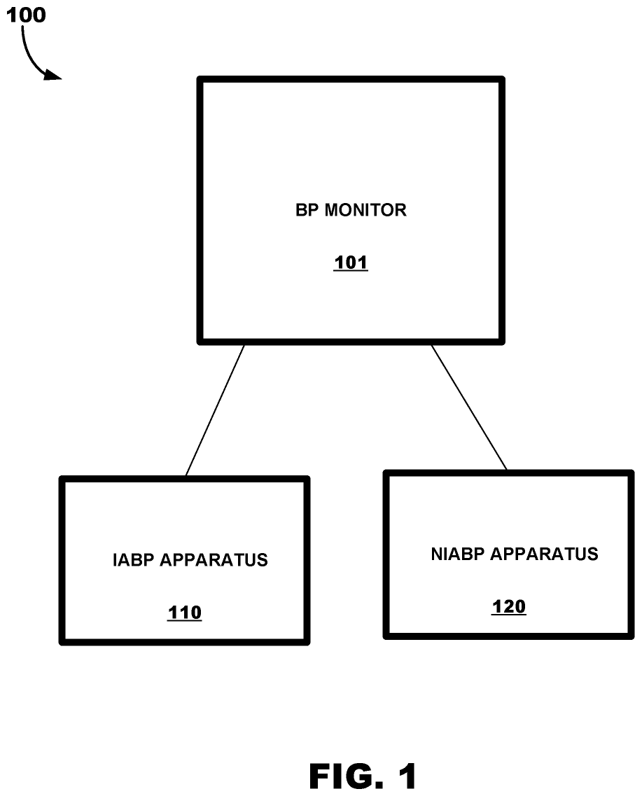

FIG. 1 illustrates an example BP monitoring system 100 in accordance with one or more embodiments set forth, described, and/or illustrated herein. The BP monitoring system 100 is configured to dynamically monitor BP performance by measuring a dynamic performance of an IABP monitor, facilitating the troubleshooting of bad dynamic measurements, and visually displaying an estimated blood pressure waveform with an indication of a range of the true dynamic waveform values given the measured invasive BP dynamic performance.

The BP monitoring system 100 comprises a BP monitor 101 operatively connected to a first BP measurement apparatus 110 and a second BP measurement apparatus 120. In one or more embodiments, the first BP measurement apparatus 110 comprises an IABP monitoring apparatus and the second BP measurement apparatus 120 comprises a NIABP monitoring apparatus.

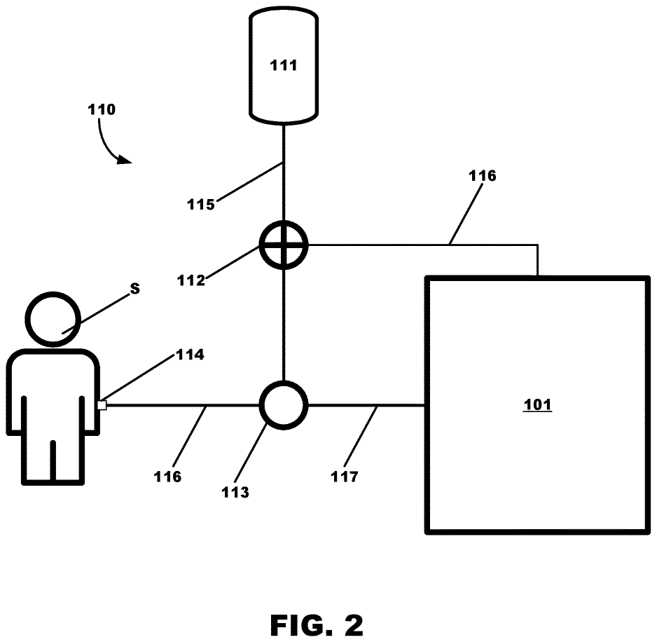

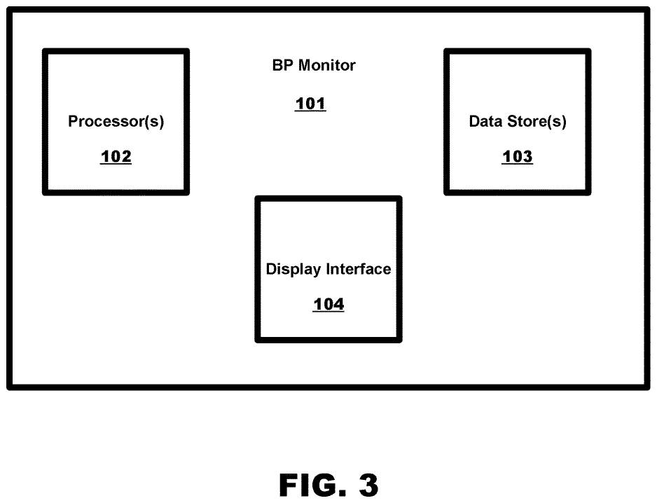

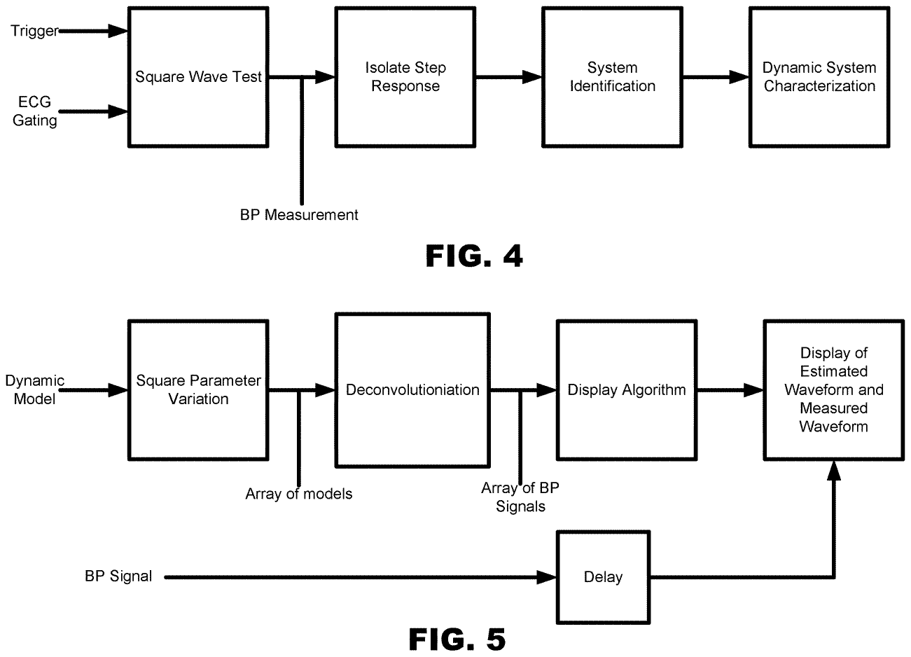

In the example embodiment of FIG. 2, the first BP measurement apparatus 110 comprises a pressurized saline reservoir 111 (which is pressurized to 300 mmHg) fluidically connected to a flush control valve 112 and an arterial pressure transducer and flush assembly 113 (that converts a pressure signal to an electronic output) via flush line tubing 115. The first BP measurement apparatus 110 further comprises an arterial catheter 114 configured for insertion in an artery site of a subject S. The arterial catheter 114 is fluidically connected to the arterial pressure transducer and flush assembly 113 via pressurized tubing 116. The electronic output from the arterial pressure transducer and flush assembly 113 is dynamically detected by the BP monitor 101, which visually displays an arterial BP waveform based on the detected electronic output.

The BP monitor 101 comprises one or more processors 102 and one or more data stores 103 having non-transitory memory operatively coupled to the one or more processors 102. As set forth, described, and/or illustrated herein, “processor” means any component or group of components that are configured to execute any of the processes described herein or any form of instructions to carry out such processes or cause such processes to be performed. The processors may be implemented with one or more general-purpose and/or one or more special-purpose processors. Examples of suitable processors include graphics processors, microprocessors, microcontrollers, DSP processors, and other circuitry that may execute software (e.g., stored on a non-transitory computer-readable medium). Further examples of suitable processors include, but are not limited to, a central processing unit (CPU), an array processor, a vector processor, a digital signal processor (DSP), a field-programmable gate array (FPGA), a programmable logic array (PLA), an application specific integrated circuit (ASIC), programmable logic circuitry, and a controller. The processors may comprise at least one hardware circuit (e.g., an integrated circuit) configured to carry out instructions contained in program code. In embodiments in which there is a plurality of processors, such processors may work independently from each other, or one or more processors may work in combination with each other.

The BP monitor 101 also comprises a display interface 104 operatively connected to the one or more processors 102. The display interface 104 may be used by a user, such as, for example, a medical clinician or professional conducting the IABP monitoring, to input one or more data input signals relating to operation of the BP monitor 101. In an example, the display interface 104 may comprise a user interface (UI), graphical user interface (GUI) such as, for example, a display, human-machine interface (HMI), or the like. Embodiments, however, are not limited thereto, and thus, this disclosure contemplates the display interface 104 comprising any suitable configuration that falls within the spirit and scope of the principles of this disclosure. The display interface 104 may also facilitate visual presentation of information/data to a user of the BP monitoring system 100. The display interface 104 may comprise one or more of a visual display or an audio display such as a microphone, earphone, and/or a speaker.

The BP monitor 101 also comprises one or more data stores 103 for storing one or more types of data. The one or more data stores 103 may comprise volatile and/or non-volatile memory. Examples of suitable data stores 103 include RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The one or more data stores 103 may be a component of the one or more processors 102, or alternatively, may be operatively connected to the one or more processors 102 for use thereby. As set forth, described, and/or illustrated herein, “operatively connected” may include direct or indirect connections, including connections without direct physical contact.

In accordance with one or more embodiments, operation of the BP monitor 101 may be implemented as computer readable program code that, when executed by the one or more processors 102, implement one or more of the various processes set forth, described, and/or illustrated herein. The BP monitor 101 may be a component of the one or more processors 102, or alternatively, may be executed on and/or distributed among other processing systems to which the one or more processors 102 are operatively connected. The BP monitor 101 may include a set of logic instructions executable by the one or more processors 102. Alternatively or additionally, the one or more data stores 103 may contain such logic instructions. The logic instructions may include assembler instructions, instruction set architecture (ISA) instructions, machine instructions, machine dependent instructions, microcode, state-setting data, configuration data for integrated circuitry, state information that personalizes electronic circuitry and/or other structural components that are native to hardware (e.g., host processor, central processing unit/CPU, microcontroller, etc.).

In accordance with an example embodiment, the non-transitory memory 103 comprises a set of instructions executable by the one or more processors 102 to cause the one or more processors 102 to initiate a flush sequence of one or more flushes of the arterial catheter 114. The one or more processors 102 may be further caused to dynamically detect a BP waveform of an arterial BP signal generated by the IABP monitoring apparatus 110, and then dynamically detect, in response to the one or more flushes, presence of damping in the detected BP waveform by applying a machine learning algorithm to conduct an analysis of the detected BP waveform which appears as a square wave stimulus.

The machine learning algorithm may be trained and used to identify or detect the presence of damping in a BP waveform of the subject S. The machine learning algorithm may comprise a first neural network, such as, for example, a deep convolution neural network, or other machine learning algorithm for purposes of identifying or detecting the presence of damping in a BP waveform. The machine learning algorithm may be trained using a BP waveform damping detection training algorithm based on BP waveform damping detection training data. The BP waveform damping detection training data may comprise data stored in the one or more data stores 103. Using the training data, the BP waveform damping detection training algorithm may train the machine learning algorithm. Upon competition of training of the machine learning algorithm, the machine learning algorithm may be implemented for purposes of identifying or the presence of damping in a BP waveform by conducting analysis of the captured data relating to the BP waveform of the subject S. The captured BP waveform data may be compared to BP waveform data of the subject S that is stored in the one or more data stores 103 to derive at a BP waveform damping detection. For example, in response to the application of the machine learning algorithm, the one or more processors 102 may be caused to generate a square wave to produce a second BP waveform, and then compare the detected BP waveform to the second blood pressure waveform of the subject S. Should the comparison confirm the presence of damping in the detected BP waveform, the one or more processors 102 may be caused to automatically initiate a second flush sequence of one or more flushes of the catheter of the invasive BP monitoring apparatus 110. For example, when an ABP damping event is detected, the one or more processors 102 may be caused to automatically issue a square-wave like flushing control signal to activate an auto-flushing control. An electric value of the auto-flushing is thus opened for a period of time (e.g., 2 s) and then stopped, so as to complete an auto-flushing operation control.

Alternatively or additionally, the machine learning algorithm may detect an ABP damping event by using the following features/signatures of the detected ABP waveform. For a given time window (e.g. 2 minutes):
1) The systolic ABP slowly decreases, from pulse to pulse,
2) The pulse ABP (i.e., the difference between the systolic and diastolic BP) slowly decreases, from pulse to pulse, to a significantly low level (e.g., 65%);
3) The mean ABP remains relatively unchanged, i.e., maintains at about the same level;
4) The diastolic ABP remains relatively unchanged, i.e., maintains at about the same level;
5) Most (e.g., 90%) of the ABP pulses have good signal quality (e.g., SQI>0.9).

The output of the machine learning algorithm is the flush necessity index (FNI) with binary value 0 or 1, with 1 for indicating a flush is required when the ABP waveform is dampened, and 0 for that the ABP waveform is not dampened.

A specific design and implementation of the ABP damping detection algorithm is as follows: Assuming, for a currently detected ABP pulse (Pᵢ), there are N pulses detected in the time window (T_{w}) prior to Pt. The ABP features from those N pulses are examined, and the following variables are defined and calculated. In accordance with one or more example embodiments, T_{w} is 2 minutes (120 s).

Percent of pulses with good signal quality in T_{w}:

$${P}_{good \_ SQI} = M / N , \qquad ( 1 )$$

where, N is the total number of pulse in the window (T_{w}), M is the number of pulses (in T_{w}) whose signal quality is good (i.e., the SQI value is above a predefined threshold, e.g. SQI>0.7).

Percent of pulses with short-term averaged systolic BP (sBPa) declining in T_{w}:

$${P}_{sBPa \_ decline} = K / N , \qquad ( 2 )$$

where, K is the number of pulses (in T_{w}) whose sBPa is lower than that of the previous pulse.

Percent of pulses with short-term averaged pulse BP (pBPa) declining in T_{w}:

$${P}_{pBPa \_ decline} = L / N ; \qquad ( 3 )$$

where, L is the number of pulses (in T_{w}) whose pBPa is lower than that of the previous pulse.

Difference of short-term averaged mean BP (mBPa) across T_{w}:

$$m B {P}_{i \_ vs \_ x} = mBP a ( i ) / mBP a ( x ) , \qquad ( 4 )$$

where, mBPa(i) is the mBPa value at the current pulse time (i), mBPa(x) is the mBPa value at time x, x=i−T_{w}.

Difference of short-term averaged diastolic BP (dBPa) across T_{w}:

$$d B {P}_{i \_ vs \_ x} = d B P a ( i ) / dBP a ( x ) ; \qquad ( 5 )$$

where, dBPa(i) is the dBPa value at the current pulse time (i), dBPa(x) is the dBPa value at time x, x=i−T_{w}.

Difference of short-term averaged pulse BP pBPa) across T_{w}:

$$p B {P}_{i \_ vs \_ x} = p B P a ( i ) / pBP a ( x ) ; \qquad ( 6 )$$

where, pBPa(i) is the pBPa value at the current pulse time (i), pBPa(x) is the pBPa value at time x, x=i−T_{w}.

The ABP flush necessity index (FNI) may be derived from the follow logic:

| IF P_{good}_SQI > thr1 | | | AND |
|---|---|---|---|
| (P_{sBPa}_decline > thr2 | OR | P_{pBPa}_decline > thr3) | AND |
| (mBPᵢ_vs_x > thr4 | OR | dBPᵢ_vs_x > thr5) | AND |
| dBPᵢ_vs_x < thr6 | | | |
| THEN | | FNI = 1; | |
| ELSE FNI = 0; | | (7), | |

where, thr1, thr2, thr3, thr4, thr5, and thr6 are appropriate thresholds, which may be obtained empirically from experimental data. In this embodiment, thr1 is chosen as 0.9, thr2 as 0.6, thr3 as 0.6, thr4 as 0.85, thr5 as 0.9, and thr6 as 0.65.

The one or more processors 102 may be caused to initiate a measurement of a pressure step signal to characterize a dynamic response of the BP monitoring system 100 by averaging the measurement of the square wave response waveform based on square wave pulse train by controlling the flush control valve 112 to pulse on and off and averaging the aligned square wave responses.

Alternatively or additionally, in response to a comparison which confirms the presence of damping in the detected blood pressure waveform, the one or more processors 102 may be caused to alert the medical clinician or professional conducting the IABP monitoring by automatically generating one or more of an audio warning signal, a video warning signal, or a haptic warning signal. For example, should the detected BP waveform be characterized as “under damped,” it is reported to the medical clinician or professional. Should the detected BP waveform be characterized as “correctly damped,” no troubleshooting guidance is necessary. Should the detected BP waveform be characterized as “overdamped,” troubleshooting guidance is supplied to the medical clinician or professional as a message reporting current and previous correlation of IABP and NIABP pressures as part of troubleshooting messages. The messages may include one or more suggestions of correcting the overdamping. Such suggestions may include, for example, reducing the number of stopcocks in the pressurized tubing 116, reducing the length of the pressurized tubing 116, tightening connections, removing air from the pressurized tubing 116, checking the fill level of the pressurized saline reservoir 111, checking pressure on the pressurized saline reservoir 111, etc.

As illustrated in FIG. 5, alternatively or additionally, in response to a comparison which confirms the presence of damping in the detected blood pressure waveform, the one or more processors 102 may be caused to calculate one or more estimated true BP waveforms without damping based on the detected blood pressure waveform. The estimated true BP waveforms may be derived via deconvolution (or Weiner filter) using a measured dynamic response and measured BP signal. The one or more processors 102 may then be caused to display of both the detected blood pressure waveform and the one or more estimated blood pressure waveforms on the display interface 104 of the blood pressure monitor 101. In particular, the detected blood pressure waveform may be displayed in a foreground of the display interface 104 and an estimated range of the one or more estimated BP waveforms due to uncertainty may be displayed in a background of the display interface 104. The detected BP waveform may be overlayed or superimposed over the one or more estimated BP waveforms. The true BP waveform may be displayed as a faint colored band behind the detected BP waveform, the width of the colored band indicating the uncertainty in the one or more estimated BP waveforms. The uncertainty of the one or more estimated BP waveforms may itself be estimated. One example estimation method is to estimate uncertainty for each estimated parameter in a second order system model and create waveforms for a grid search of all those parameter variations. A Monte Carlo approach may be used instead of a grid search.

Alternatively or additionally, in response to a comparison which confirms the presence of damping in the detected BP waveform, the one or more processors 102 may be caused to automatically initiate an NIABP measurement of the subject S. The NIABP measurement may not be initiated in the event one or more recent NIABP measurements of the subject S is available and stored in the one or more data stores 103.

In accordance with one or more example embodiments, the BP monitoring system 100 dynamically monitors BP performance by considering one or more distinguishable patterns in an IABP waveform that are associated with damping. The machine learning algorithm is applied to dynamically analyze the IABP waveform in real-time to automatically detect the one or more distinguishable patterns. Once damping is detected, the one or more processors 102 are caused to send a first control signal to automatically initiate a fast-flushing sequence. The machine learning algorithm continues to analyze the IABP waveform after the fast-flushing sequence and evaluates whether the detected, dampened IABP waveform is resolved (i.e., returned to normal). Should the dampened IABP waveform be resolved after the fast-flushing sequence, the IABP monitoring process continues.

In accordance with one or more example embodiments, the automatic fast-flushing sequence may be conducted via a flushing apparatus comprising a computer controlled electric fluid value in the flush line tubing 115. Upon receiving an activation signal from the one or more processors 102, the BP monitoring system 100 makes a timed (in terms of the time and duration) fast flush.

The BP monitor 101 is configured to track the ABP waveform immediately after an automatic flushing sequence, and then determines whether the dampened ABP waveform is recovered, i.e., the flushing operation is effective. Recovery of the ABP waveform may be indicated by one or more distinguishable signatures, e.g., the systolic BP and pulse BP values are significantly enlarged, and the maximum ABP waveform slope is significantly increased.

In accordance with one or more embodiments, detection of ABP recovery by the BP monitor 101 after implementation of the automatic flushing sequence may be conducted via the one or more processors 102 initiating a flush effectiveness assessment (FEA) algorithm. The FEA algorithm tracks certain ABP waveform features immediately following the automatic flushing sequence, and then compares them to those same features taken immediately before the automatic flushing sequence to determine if the ABP waveform is recovered.

A specific design and implementation of the FEA algorithm by the one or more processors 102 may be executed as follows. Assuming that the automatic flushing sequence start time is Fₒₙ. The following ABP features before the flush sequence are calculated from those ABP pulses in a period of T_{b2} (e.g., 10 s) which is located at T_{b1} (e.g., 2 s) before Fₒₙ:
sBPa_before_flush, which is the averaged systolic ABP in T_{b2};
pBPa_before_flush, which is the averaged pulse ABP in T_{b2};
mxSLPa_before_flush, which is the averaged maximum ABP waveform slope in T_{b2}, where only those ABP pulses in T_{b2} with good signal quality (e.g., SQI>0.9) are taken into account. In this embodiment, T_{b2} is chosen as 10 s; T_{b1} as 2 s, and SQI threshold as 0.9.

Assuming that the automatic flushing sequence end time is F_{off}. The following ABP features after the flush are calculated from the ABP pulses in the period of Tₑ₂ (e.g., 10 s) which is located at Tₑ₁ (e.g., 3 s) after F_{off}:
sBPa_after_flush, which is the averaged systolic ABP in Tₑ₂;
pBPa_after_flush, which is the averaged pulse ABP in Tₑ₂;
mxSLPa_after_flush, which is the averaged maximum ABP waveform slope in Tₑ₂, where, only those ABP pulses in Tₑ₂ with good signal quality (e.g. SQI>0.9) are taken into account. In accordance with one or more example embodiments, Tₑ₂ is chosen as 10 s; Tₑ₁ as 3 s, and SQI threshold as 0.9.

A flush effectiveness index (FEI) may be derived from the following logic:

| FEI = 0; | | |
|---|---|---|
| IF pABPa_after_flush / pABPa_before_flush | > r1 | AND |
| sABPa_after_flush / sABPa_before_flush | > r2 | AND |
| mxSLPa_after_flush / mxSLPa_before_flush | > r3 | AND |
| THEN FEI = 1; (for 2s) | | |
| ELSE FEI = −1; (for 2s) | | (8), |

where, r1, r2, and r3 are appropriate (ratio) thresholds, which may be obtained empirically from experimental data. In accordance with one or more example embodiments, r1 is chosen as 1.5, r2 as 1.2, and r3 as 2.0.

The FEI value is initialized as 0. Should the automatic flushing sequence be effective (i.e., ABP signal recovered), the FEI receives a value of “1” (for 2 s, and then returns to a value of 0, in order to visually illustrate the result). Should, on the other hand, the automatic flushing sequence not be successful, the FEI receives a value of “−1” (for 2 s, and the returns to a value of 0, in order to visually illustrate the result). The judgement is made at the time (Tₑ₁+Tₑ₂) after F_{off}. This short delay is necessary due to the need of a reasonable period of time to reliably obtain the ABP features after the automatic flushing sequence. The skip windows T_{b1} and Tₑ₁, are introduced for excluding those ABP waveforms which are very close to (and thus, might be disturbed by) the automatic flushing sequence.

The FEI is calculated shortly after the end of each automatic flushing sequence.

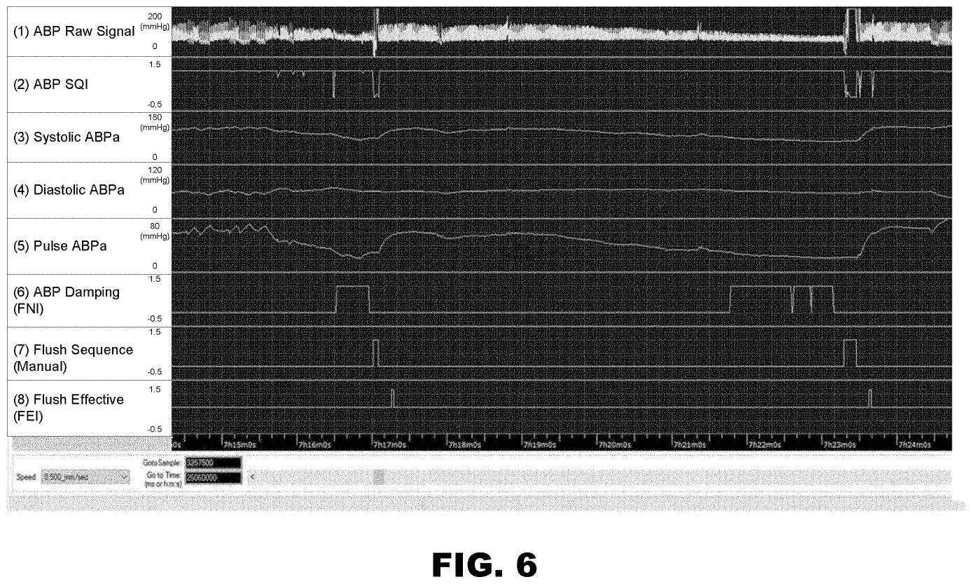

In the illustrated example of FIG. 6, to illustrate the FEI result, a flushing signal was generated, as illustrated in panel (7) of FIG. 6, according to the manual flushing operations on the ABP record for a skilled ICU the medical clinician or professional. The flushing signal comprises a square-wave function, with its non-zero values corresponding to the manual flushing operations (as identified by the saturated ABP signal illustrated in Panel 1) for the medical clinician or professional. The proposed FEA algorithm executed by the one or more processors 102 takes in the onset time and offset time of each flushing operation, and produced the FEI value at (Tₑ₁+Tₑ₂) after the flush offset time for this flush, as seen in Panel 8. The FEI value lasts for a duration of 2 s and returns to zero for visually observing the result of the flush effectiveness assessment. As seen in Panel 8, both the flushes are correctly assessed as effective.

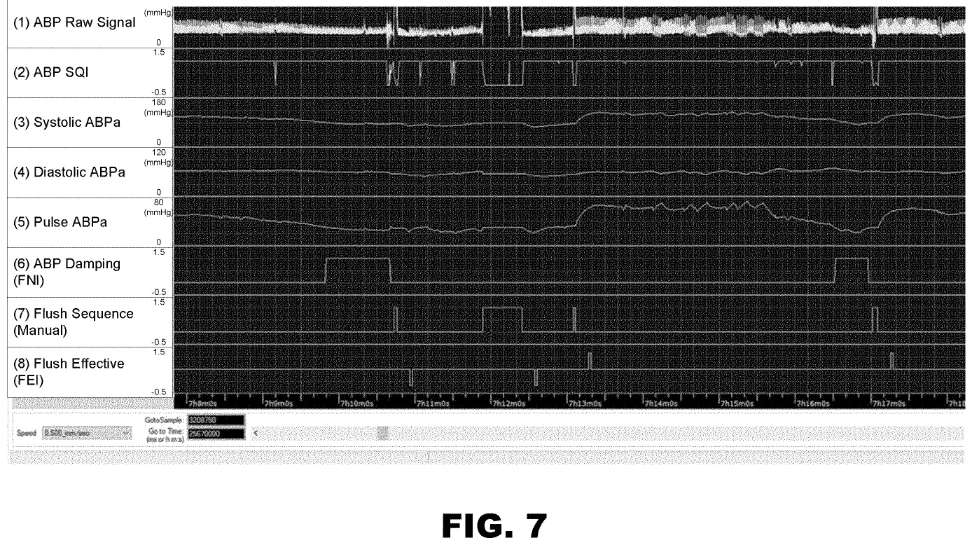

In the illustrated example of FIG. 7, a plurality of flushes was performed for the IABP damping detection and flush effectiveness assessment. For the first IABP damping event, it is correctly detected by the BP monitor 101, as indicated by the FNI value in Panel 6, also preceding a visual observation by the medical clinician or professional. The flush sequence comprises three manual flushes performed by the medical clinician or professional (as indicated in Panel 7). The first two flushes were not effective, and the third flush was successful (i.e., effective). The proposed FEA algorithm executed by the one or more processors 102 correctly assessed the flushing effectiveness, by producing FEI with a value of −1 for the first two flushes and FEI with a value of 1 for the third flush (as indicated in Panel 8). The second IABP damping event is correctly detected by the BP monitor 101 before manual identification by the medical clinician or professional. The manual flushing operation (at around 07:17:00) is correctly assessed as effective (as FEI has a value of 1).

In accordance with one or more example embodiments, the BP monitoring system 100 may be configured to identify the detected IABP data stream as “questionable,” thereby suppressing the generation of an alarm based on a false positive physiological alarm pursuant to its logic.

Should the ABP waveform be recovered after an automatic flushing sequence, the IABP monitoring continues. In the event the IABP is not recovered, a subsequent automatic flushing sequence may be performed. Should the number of the flushes in the sequence exceed a predefined or predetermined numeric value M (e.g., M=3) and the IABP waveform is still not recovered, the one or more processors 102 may cause the generation of an alarm (e.g., one or more of an audio warning signal, a video warning signal, or a haptic warning signal) to alert the medical clinician or professional by indicating that the pressurized tubing 116 has an overdamping problem and needs manually intervention to resolve the damping issue.

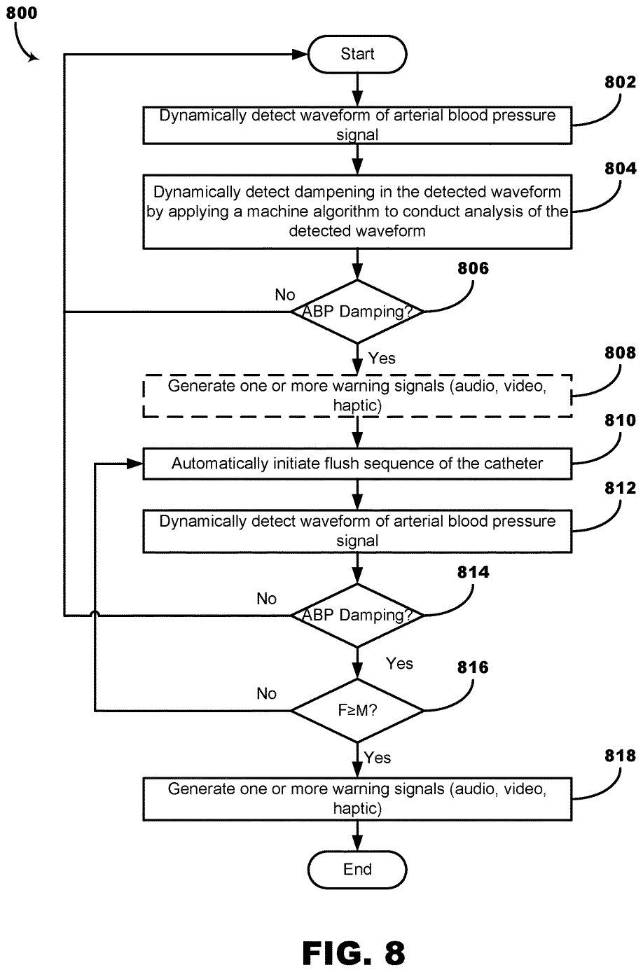

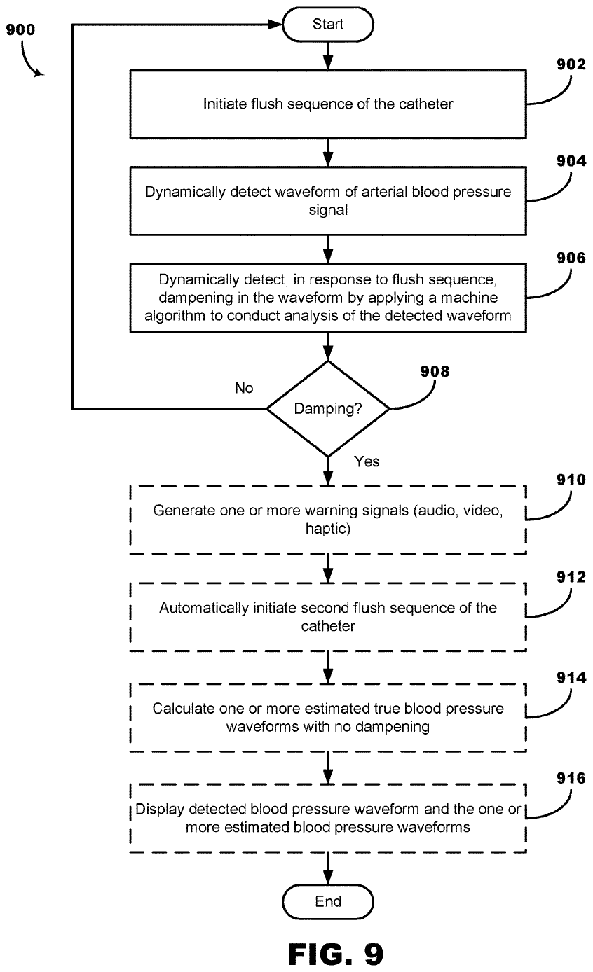

The illustrated examples of FIGS. 8 and 9 respectively represent a flowchart of an example computer-implemented method 800, 900 of dynamically monitoring a blood pressure measurement. The flowchart of each computer-implemented method 800, 900 may be implemented by one or processors 102 of the BP monitor 101.

In particular, each example computer-implemented method 800, 900 may be implemented as one or more modules in a set of logic instructions stored in a non-transitory machine- or computer-readable storage medium such as random access memory (RAM), read only memory (ROM), programmable ROM (PROM), firmware, flash memory, etc., in configurable logic such as, for example, programmable logic arrays (PLAs), field programmable gate arrays (FPGAs), complex programmable logic devices (CPLDs), in fixed-functionality hardware logic using circuit technology such as, for example, application specific integrated circuit (ASIC), complementary metal oxide semiconductor (CMOS) or transistor-transistor logic (TTL) technology, or any combination thereof.

Software executed by the BP monitor 101 provides functionality described or illustrated herein. In particular, software executed by the one or processors 102 is configured to perform one or more processing blocks of each example computer-implemented method 800, 900 or provides functionality set forth, described, and/or illustrated herein.

In the illustrated example computer-implemented method 800 of FIG. 8, illustrated process block 802 includes dynamically detecting a BP waveform of an ABP signal generated by an IABP monitoring apparatus.

The computer-implemented method 800 may then proceed to illustrated process block 804, which includes dynamically detecting presence of damping in the detected BP waveform by applying a machine learning algorithm to conduct an analysis of the detected BP waveform which appears as a square wave stimulus.

The computer-implemented method 800 may then proceed to illustrated process block 806, which includes making a determination of whether there is ABP damping in the detected BP waveform.

If “No,” i.e., there is no detection of damping, the computer-implemented method 800 may then return to start.

If “Yes,” the computer-implemented method 800 may then proceed to illustrated process block 808, which includes alerting the medical clinician or professional conducting the IABP monitoring by generating one or more of an audio warning signal, a video warning signal, or a haptic warning signal.

Alternatively or additionally, if “Yes,” the computer-implemented method 800 may then proceed to illustrated process block 810, which includes automatically initiating a flush sequence of one or more flushes of a catheter of the IABP monitoring apparatus.

The computer-implemented method 800 may then proceed to illustrated process block 812, which includes dynamically detecting, in response to the flush sequence, the BP waveform.

The computer-implemented method 800 may then proceed to illustrated process block 814, which includes making a determination of whether there is ABP damping in the detected BP waveform.

If “No,” i.e., there is no detection of damping, the computer-implemented method 800 may then return to start.

If “Yes,” the computer-implemented method 800 may then proceed to illustrated process block 816, which includes a determination of whether the overall number of flushes F is greater than a predetermined threshold number M.

If “No,” the computer-implemented method 800 may then return to illustrated process block 810 to conduct another flush sequence of one or more flushes.

If “Yes,” the computer-implemented method 800 may then proceed to illustrated process block 818, which includes alerting the medical clinician or professional conducting the IABP monitoring by generating one or more of an audio warning signal, a video warning signal, or a haptic warning signal. The computer-implemented method 800 can terminate or end after execution of illustrated process block 818.

In the illustrated example computer-implemented method 900 of FIG. 7, illustrated process block 902 includes initiating a flush sequence of one or more flushes of a catheter of an IABP monitoring apparatus.

The computer-implemented method 900 may then proceed to illustrated process block 904, which includes dynamically detecting a BP waveform of an ABP signal generated by the IABP monitoring apparatus.

The computer-implemented method 900 may then proceed to illustrated process block 906, which includes dynamically detecting presence of damping in the detected BP waveform by applying a machine learning algorithm to conduct an analysis of the detected BP waveform which appears as a square wave stimulus.

The computer-implemented method 900 may then proceed to illustrated process block 908, which includes making a determination of whether there is ABP damping in the detected BP waveform.

If “No,” i.e., there is no detection of damping, the computer-implemented method 900 may then return to start.

If “Yes,” the computer-implemented method 900 may then proceed to illustrated process block 910, which includes alerting the medical clinician or professional conducting the IABP monitoring by generating one or more of an audio warning signal, a video warning signal, or a haptic warning signal.

Alternatively or additionally, if “Yes,” the computer-implemented method 900 may then proceed to illustrated process block 912, which includes automatically initiating a flush sequence of one or more flushes of a catheter of the IABP monitoring apparatus.

Alternatively or additionally, if “Yes,” the computer-implemented method 900 may then proceed to illustrated process block 914, which includes calculating one or more estimated true BP waveforms (with no damping) based on the detected BP waveform.

The computer-implemented method 900 may then proceed to illustrated process block 916, which includes displaying the detected BP waveform (in a foreground of a display interface) and an estimated range of the calculated one or more estimated BP waveforms due to uncertainty (in a background of the display interface). The computer-implemented method 900 can terminate or end after execution of illustrated process block 916.

The terms “coupled,” “attached,” or “connected” may be used herein to refer to any type of relationship, direct or indirect, between the components in question, and may apply to electrical, mechanical, fluid, optical, electromagnetic, electromechanical or other connections. In addition, the terms “first,” “second,” etc. are used herein only to facilitate discussion, and carry no particular temporal or chronological significance unless otherwise indicated.

Those skilled in the art will appreciate from the foregoing description that the broad techniques of the embodiments of the present invention can be implemented in a variety of forms. Therefore, while the embodiments set forth, described, and/or illustrated herein have been described in connection with particular examples thereof, the true scope of the embodiments should not be so limited since other modifications will become apparent to the skilled practitioner upon a study of the drawings, specification, and claims.

1. A system, comprising:
an invasive arterial blood pressure monitoring apparatus; and
a blood pressure monitor operatively connected to the invasive arterial blood pressure monitoring apparatus, the blood pressure monitor including one or more processors and a non-transitory memory operatively coupled to the one or more processors comprising a set of instructions executable by the one or more processors to cause the one or more processors to:
initiate a first flush sequence of one or more flushes of a catheter of the invasive arterial blood pressure monitoring apparatus;
dynamically detect a blood pressure waveform of an arterial blood pressure signal generated by the invasive arterial blood pressure monitoring apparatus; and
dynamically detect, responsive to the first flush sequence, presence of damping in the detected blood pressure waveform by applying a machine learning algorithm to conduct an analysis of the detected blood pressure waveform which appears as a square wave stimulus.

2. The system of claim 1, wherein the set of instructions cause the one or more processors to generate, in response to the application of the machine learning algorithm, a square wave to produce a second blood pressure waveform.

3. The system of claim 2, wherein the set of instructions cause the one or more processors to compare the detected blood pressure waveform to the second blood pressure waveform.

4. The system of claim 3, wherein the set of instructions cause the one or more processors to automatically initiate, in response to a comparison which confirms the presence of damping in the detected blood pressure waveform, a second flush sequence of one or more flushes of the catheter of the invasive blood pressure monitoring apparatus.

5. The system of claim 3, wherein the set of instructions cause the one or more processors to generate, in response to a comparison which confirms the presence of damping in the detected blood pressure waveform, one or more of an audio warning signal, a video warning signal, or a haptic warning signal.

6. The system of claim 3, wherein the set of instructions cause the one or more processors to calculate, in response to a comparison which confirms the presence of damping in the detected blood pressure waveform, one or more estimated true blood pressure waveforms without damping based on the detected blood pressure waveform.

7. The system of claim 6, wherein the set of instructions cause the one or more processors to display, on a display interface of the blood pressure monitor, of the detected blood pressure waveform in a foreground of the display interface and a range of the one or more estimated blood pressure waveforms in a background of the display interface.

8. A computer-implemented method of dynamically monitoring a blood pressure measurement, comprising:
initiating a first flush sequence of one or more flushes of a catheter of an invasive arterial blood pressure monitoring apparatus;
dynamically detecting a blood pressure waveform of an arterial blood pressure signal generated by the invasive arterial blood pressure monitoring apparatus; and
dynamically detecting, responsive to the first flush sequence, presence of damping in the detected blood pressure waveform by applying a machine learning algorithm to conduct an analysis of the detected blood pressure waveform which appears as a square wave stimulus.

9. The computer-implemented method of claim 8, wherein dynamically detecting damping comprises applying the machine learning algorithm to generate a square wave that produces a second blood pressure waveform.

10. The computer-implemented method of claim 9, wherein the analysis comprises comparing the detected blood pressure waveform to the second blood pressure waveform.

11. The computer-implemented method of claim 10, further comprising automatically initiating, in response to a comparison which confirms presence of damping in the detected blood pressure waveform, a second flush sequence of one or more flushes of the arterial catheter of the invasive arterial blood pressure monitoring apparatus.

12. The computer-implemented method of claim 10, further comprising generating, in response to a comparison which confirms presence of damping in the detected blood pressure waveform, one or more of an audio warning signal, a video warning signal, or a haptic warning signal.

13. The computer-implemented method of claim 10, further comprising calculating, in response to a comparison which confirms presence of damping in the detected blood pressure waveform, one or more estimated blood pressure waveforms without damping based on the detected blood pressure waveform.

14. The computer-implemented method of claim 13, further comprising causing, on a display interface, a display of the detected blood pressure waveform in a foreground of a display interface and a range of the one or more estimated blood pressure waveforms in a background of the display interface.

15. An apparatus, comprising:
a blood pressure monitor including one or more processors and a non-transitory memory operatively coupled to the one or more processors comprising a set of instructions executable by the one or more processors to cause the one or more processors to:
initiate a first flush sequence of one or more flushes of a catheter of an invasive arterial blood pressure monitoring apparatus;
dynamically detect a blood pressure waveform of an arterial blood pressure signal generated by the invasive arterial blood pressure monitoring apparatus; and
dynamically detect, responsive to the first flush sequence, presence of damping in the detected blood pressure waveform by applying a machine learning algorithm to conduct an analysis of the detected blood pressure waveform which appears as a square wave stimulus.

16. The apparatus of claim 15, wherein the machine learning algorithm generates a square wave to produce a second blood pressure waveform.

17. The apparatus of claim 15, wherein the set of instructions cause the one or more processors to compare the detected blood pressure waveform to the second blood pressure waveform.

18. The apparatus of claim 15, wherein the set of instructions cause the one or more processors to automatically initiate, in response to a comparison which confirms presence of damping in the detected blood pressure waveform, a second flush sequence of one or more flushes of the arterial catheter of the invasive arterial blood pressure monitoring apparatus.

19. The apparatus of claim 15, wherein the set of instructions cause the one or more processors to generate, in response to a comparison which confirms the presence of damping in the detected blood pressure waveform, one or more of an audio warning signal, a video warning signal, or a haptic warning signal.

20. The apparatus of claim 15, wherein the set of instructions cause the one or more processors to:
calculate, in response to a comparison which confirms the presence of damping in the detected blood pressure waveform, one or more estimated blood pressure waveforms without damping based on the detected blood pressure waveform, and
cause, on a display interface, a display of the detected blood pressure waveform in a foreground of a display interface and a range of the estimated blood pressure waveform in a background of the display interface.
